(19) United States
(12) Patent Application Publication
(10) Pub. No.: US 2022/0208320 A1
(43) Pub. Date: Jun. 30, 2022

(54) System and Method for Displaying Physiological Information

(71) Applicant: Tricog Health Pte Ltd, Singapore (SG)

(72) Inventors: Charit Bhograj, Bangalore (IN); Udayan Dasgupta, Bangalore (IN); Abhinav Gujjar, Bangalore (IN); Manmay Nakhashi, Vadodara (IN); Suresh Velusamy, Tiruppur (IN); Achuth Pv, Calicut (IN); Tharun Kumar, Challakere (IN); Zainul Charbiwala, Bengaluru (IN)

(21) Appl. No.: 17/604,166

(22) PCT Filed: Apr. 15, 2020
(86) PCT No.: PCT/SG2020/050234

§ 371 Date: Oct. 15, 2021

(30) Foreign Application Priority Data
Apr. 16, 2019 (IN) 2019 41015294

(51) Int. Cl. G16H 10/60; A61B 5/00

(52) U.S. Cl. CPC G16H 10/60; A61B 5/7275; A61B 5/7475; A61B 5/742

(57) ABSTRACT

A system and method for displaying physiological information is disclosed. The system includes a server to receive a first compressed data file associated with a physiological parameter of a person, decompress the first compressed data file, and analyze the decompressed data file to generate or derive a set of information associated with the person, and a client device to receive the first compressed data file, and communicate with the server to receive the set of information for display on a user interface. The server compresses the set of information to generate a second compressed data file, and sends the second compressed data file to the client device. The client device decompresses the received second compressed data and a part of the first compressed data file, and displays at least a part of the decompressed data on the user interface.

### CROSS-REFERENCE TO RELATED APPLICATIONS

This application is the United States national phase of International Application No. PCT/SG2020/050234 filed Apr. 15, 2020, and claims priority to Indian Patent Application No. 201941015294 filed Apr. 16, 2019, the disclosures of which are hereby incorporated by reference in their entirety.

### BACKGROUND OF THE INVENTION

### Field of the Invention

The present disclosure relates to a system and method for displaying physiological information, in particular in the display of physiological information associated with a person or individual.

### Description of Related Art

The following discussion of the background to the invention is intended to facilitate an understanding of the present invention only. It may be appreciated that the discussion is not an acknowledgement or admission that any of the material referred to was published, known or part of the common general knowledge of the person skilled in the art in any jurisdiction as at the priority date of the invention.

Heart diseases, for example, arrhythmia, may be discovered by monitoring heart conditions in which the heart rate is irregular or beyond the normal limits, for example too fast or too slow. While some types of arrhythmia are asymptomatic, others may cause palpitations, lightheadedness or even fainting episodes. Some arrhythmias may lead to serious consequences such as stroke, heart failure or cardiac arrest. The arrhythmias may often occur only for short periods of time and may coincide with physical or emotional stress.

However, due to the characteristic of the arrhythmia, it may be difficult to find out the seriousness of the arrhythmia by an observation in a short term. Hence, a person with arrhythmia may be required to monitor his electrocardiography (ECG) data continuously in a long term. Medical devices such as, for example, an ECG machine, may be used to continuously monitor the person's heart conditions in the long term, and create bulk of ECG waveforms as a result.

However, it may be time consuming for doctors, for example, cardiac specialists, to read and analyse the bulk of data to determine a diagnosis manually. To address the problem, the doctors use their computing devices. While the computing devices are useful to aid the diagnosis, the doctors may face a difficulty in storing and processing the bulk of data using their computing device, since the amount of data that needs to be stored and processed is large relative to the available resource of their computing device.

In light of the above, there exists a need for reducing the resource requirements of the doctor's computing device for storing and processing the data. There exists a further need to provide a solution that meets the mentioned needs or alleviates the challenges at least in part.

### SUMMARY OF THE INVENTION

The principal object of the invention is to provide a system for displaying physiological information of a person.

Another object of the invention is to provide a server to receive, decompress and analyze a compressed data file associated with the physiological information of the person.

Yet another object of the invention is to provide a method of generating a set of information associated with the user.

Yet another object of the invention is to provide a client device to display the set of information on a user interface.

Yet another object of the invention is to provide a method of compressing and decompressing data file and display the decompressed data file on the user interface.

### BRIEF DESCRIPTION OF THE DRAWINGS

This invention is illustrated in the accompanying drawings, throughout which, reference letters indicate corresponding parts in the various figures.

The embodiments herein will be better understood from the following description with reference to the drawings, in which:

FIG. 1 shows a block diagram in accordance with some embodiments of the present invention.

FIG. 2 shows another block diagram in accordance with some embodiments of the present invention.

FIG. 3 shows a flow diagram in accordance with some embodiments of the present invention.

FIGS. 4 to 6 show examples of information displayed in the client device in accordance with some embodiments of the present invention.

Other arrangements of the invention are possible and, consequently, the accompanying drawings are not to be understood as superseding the generality of the preceding description of the invention.

### DESCRIPTION OF THE INVENTION

A systematic way of reducing resource requirements of a device (hereinafter referred to as “client device”) used to aid the doctor's diagnosis is envisaged.

The technical solution is provided in the form of a system and method for displaying physiological information. In particular, a server and a client device receive a compressed data file associated with a physiological parameter, for example ECG waveforms, of a person (hereinafter referred to as a “first compressed data file”). The server may decompress the first compressed data file, analyze the decompressed data file, and generate or derive a set of information, for example a set of medical information, associated with the person. The server may compress the set of information to be in a compressed format (hereinafter referred to as a “second compressed data file”), and send the second compressed data file to the client device. The client device may decompress the second compressed data file to display the set of information on the user interface. In addition, the client device may decompress the first compressed data file, only on as-needed basis. In some embodiments, the client device may decompress a part of the first compressed data file which is associated with a selected part of the set of information, and display at least a part of data corresponding to the part of the first compressed data file on the user interface.

In this manner, the resource requirements, for example, disk space, memory, processing speed, of the client device may be reduced. The invention may allow the client device to run on lower-end computers, whereas sophisticated algorithms which require large memory and computation may run on the server.

With the proliferation of communication devices such as mobile phones, the ECG waveforms may increasingly be viewed and diagnosed remotely from the communication devices. The invention may also allow the client device to reduce the computational requirements and minimize transmission overhead. As such, a remote diagnosis can be achieved even in the simple communication devices as the client device.

The embodiments herein and the various features and advantageous details thereof are explained more fully with reference to the non-limiting embodiments that are illustrated in the accompanying drawings and/or detailed in the following description. Descriptions of well-known components and processing techniques are omitted so as to not unnecessarily obscure the embodiments herein. The examples used herein are intended merely to facilitate an understanding of ways in which the embodiments herein may be practised and to further enable those of skill in the art to practice the embodiments herein. Accordingly, the examples should not be construed as limiting the scope of the embodiments herein.

In the context of the present invention, there is a system for displaying physiological information comprising: a server arranged to receive a first compressed data file associated with a physiological parameter of a person, decompress the first compressed data file, and analyse the decompressed data file to generate or derive a set of information associated with the person; and a client device configured to receive the first compressed data file, and communicate with the server to receive the set of information for display on a user interface; wherein the server is configured to compress the set of information to generate a second compressed data file, and send the second compressed data file to the client device; and the client device is configured to receive the second compressed data file from the server, decompress the second compressed data file to display the set of information on the user interface, and decompress a part of the first compressed data file which is associated with a selected part of the set of information, to display at least a part of data corresponding to the part of the first compressed data file on the user interface.

In some embodiments, the analysis of the decompressed data file includes a comparison between at least one feature extracted from the decompressed data file and a predetermined physiological parameter.

In some embodiments, the analysis of the decompressed data file includes a detection of a predetermined event in the decompressed data file, and the set of information contains at least one annotation associated with the detected predetermined event.

In some embodiments, the detection of the predetermined event is performed based on presence or absence of at least one feature estimated from the decompressed data file.

In some embodiments, the detection of the predetermined event includes processing of the decompressed data file and a classification of the decompressed data file as an instance of the predetermined event.

In some embodiments, the analysis of the decompressed data file includes an analysis of trend in an activity of the person's body associated with the physiological parameter, and the set of information contains the analyzed trend.

In some embodiments, the analysis of the decompressed data file includes a classification of the decompressed data file into at least one preliminary group of medical condition based on the detected predetermined event and/or the analyzed trend.

In some embodiments, the client device is operable to split the first compressed data file into a plurality of groups based on a predetermined time interval.

In some embodiments, the client device is operable to display a plurality of decompressed data strips on the user interface.

In some embodiments, if a data strip among the plurality of decompressed data strips is selected, the client device is operable to display an extended data strip corresponding to the selected decompressed data strip on the user interface.

In some embodiments, if a region in the extended data strip is selected, the client device is operable to decompress a part of the first compressed data file corresponding to the selected region of the extended data strip and display at least a part of decompressed waveforms corresponding to the selected region on the user interface.

In some embodiments, if a region in the decompressed waveforms is selected, the client device is operable to display extended waveforms corresponding the selected region of the decompressed waveforms on the user interface.

In some embodiments, the annotation is displayed on the decompressed waveforms and/or extended waveforms.

In some embodiments, if at least one of the first or second compressed data file is not compatible with the client device, the client device is operable to inform the server, and the server is operable to convert a format of the at least one of the first or second compressed data file to be compatible with the client device.

In some embodiments, if the client device obtains another data file which is not compatible with the client device, the client device is operable to send the another data file to the server, and the server is operable to convert the another data file to be compatible with the client device.

In some embodiments, if the client device receives an input to change the information, the client device updates the information based on the input and sends the updated information to the server.

In another aspect, there is a method for displaying physiological information comprising: receiving, at a server and a client device, a first compressed data file associated with a physiological parameter of a person; decompressing, at the server, the first compressed data file; analysing, at the server, the decompressed data file to generate or derive a set of information associated with the person; compressing, at the server, the set of information to generate a second compressed data file; sending, at the server, the second compressed data file to the client device; receiving, at the client device, the second compressed data file from the server; decompressing, at the client device, the second compressed data file to display the set of information on the user interface; and decompressing, at the client device, a part of the first compressed data file which is associated with a selected part of the set of information, to display at least a part of data corresponding to the part of the first compressed data file on the user interface.

Other aspects of the invention may be apparent to those of ordinary skilled in the art upon review of the following description of specific embodiments of the invention in conjunction with the accompanying drawings.

Throughout the specification, unless the context requires otherwise, the word “comprise” or variations such as “comprises” or “comprising”, will be understood to imply the inclusion of a stated integer or group of integers but not the exclusion of any other integer or group of integers.

Furthermore, throughout the specification, unless the context requires otherwise, the word “include” or variations such as “includes” or “including”, will be understood to imply the inclusion of a stated integer or group of integers but not the exclusion of any other integer or group of integers.

Throughout the description, a communication device may include, but not be limited to, smartphone, desktop computer, laptop, tablet computer and wearable device, in particular intelligent wearable device such as smart watch, smart glasses or mobile virtual reality headset.

Referring now to the drawings, where similar reference characters denote corresponding features consistently throughout the figures, there are shown preferred embodiments.

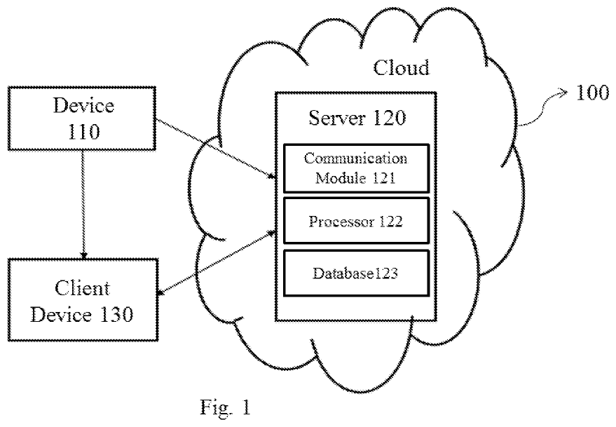

FIG. 1 shows a block diagram in accordance with some embodiments of the present invention. The system 100 may comprise a server 120 and a client device 130. The system 100 may further comprise a device 110 arranged in signal or data communication with the server 120 and/or the client device 130.

The system 100 may include a software platform for use by a user, for example a doctor, for a diagnosis, for example an assessment of arrhythmias, from historical data, for example electrocardiography (ECG) data, of a person. The person or individual may include a human being and/or mammalian animal.

The system 100 may include a cloud connected platform which comprises a client-side application (referred to as “client device 130”) coupled with a server-side software (referred to as “server 120”). The server 120 may use at least one algorithm to analyse and process a dataset. For example, the dataset may be a set of data, for example ECG waveform. The client device 130 may be used by the doctor to manage the person's records, view the dataset, for example the ECG waveforms, with a set of information generated by the server 120, and prepare a final report in relation to the person. It may be appreciated that the client device 130 may be a software application and/or a communication device.

The device 110 may include, but not be limited to, a home medical equipment used for a person at home or other facilities. The home medical equipment may include, but not be limited to, an ECG machine such as a Holter monitor which measures an electrical activity of the person's heart to show whether the heart is working normally. The device 110 may collect the dataset associated with a physiological parameter of a person. For example, the ECG machine may collect the dataset associated with the rhythm and activity of the person's heart. In some embodiments, the ECG machine may output the collected dataset on a screen and/or a paper.

The device 110 can compress the dataset to be in a compressed data format and generate a compressed data file (hereinafter referred to as a “first compressed data file”). In some embodiments, the device 110 can send the first compressed data file to the server 120 and/or the client device 130. In some other embodiments, the device 110 can send the first compressed data file to the server 120 and then the server 120 can send the first compressed data file to the client device 130.

In some embodiments, the first compressed data file may be sent at the end of the recording or at predetermined intervals, for example regular intervals such as every hours. In some other embodiments, the first compressed data file may be sent if the environment satisfies a predetermined criteria. For example, when the network is available, the first compressed data file may be sent. In some other embodiments, on a request, the first compressed data file may be sent. In some other embodiments, when some predetermined event occurred, the first compressed data file may be sent. In some other embodiments, when at least two above conditions are met, the first compressed data file may be sent.

In some embodiments, the person may directly use the device 110 so that the device 110 may collect the dataset associated with the physiological parameter of the person. In some other embodiments, another person, for example a doctor, a nurse, a technician and/or a guardian, may assist the person to use the device 110, and thereby the device 110 may collect the dataset associated with the physiological parameter of the person.

In some embodiments, the device 110 may include a communication device, for example the person's communication device. The communication device as the device 110 may collect the dataset associated with the physiological parameter of the person. For example, the communication device may collect the dataset associated with the rhythm and activity of the person's heart from the ECG machine, compress the dataset to generate the first compressed data file, and send out the first compressed data file to the server 120.

In some embodiments, an API (Application programming interface) may be used to upload the dataset in the form of file(s) to the server 120. For example, the API may be used in case of real-time monitoring of the dataset, for example ECG waveforms, where the ECG waveforms reach the server 120 in parts and the ECG waveforms and the analysis need to be viewed at the client device 130. In this case, the uploaded file(s) may be converted to the format which is compatible with client device 130. In this manner, the uploaded file(s) may be converted to the first compressed data file. Thereafter, the client device 130 may download the first compressed data file and other processing may proceed in the usual fashion.

The server 120 may comprise a communication module 121, a processor 122 and a database 123. It may be appreciated that in some other embodiments, the communication module 121 and the processor 122 may be integrated. Some embodiments can be implemented in or supported by a cloud network infrastructure. The server 120 may be a cloud server which is built, hosted and delivered through a cloud computing platform over a communication network, for example Internet. The cloud server may be accessed remotely from a plurality of users including the person and the doctor.

The communication module 121 may include one or more modules or units which permit wired communications and/or wireless communications with the device 110 and/or the client device 130 to be described below. For example, the communication module 121 receives ECG waveforms from the device 110 and sends information generated by the processor 122 to the client device 130. The information may include a classification result of the dataset into at least one preliminary group of medical condition determined by the processor 122 and/or one or more annotations inserted by the processor 122 to be described below. The information may be in the form of audio signal, video signal, text signal, multimedia signals, or combination thereof. The information may also include various formats of dataset.

The processor 122 is operable to decompress the first compressed data file, and analyse the decompressed data file using at least one algorithm to generate or derive a set of information, for example a set of medical information, associated with the person. In an example, the decompressed data file may include the dataset. In another example, the decompressed data file may be the same as the dataset. The analysis of the decompressed data file may include a comparison between at least one feature extracted from the decompressed data file and a predetermined physiological parameter. The predetermined physiological parameter may be stored in the database 123. For the analysis of the decompressed data file, the processor 122 can extract the at least one feature from the decompressed data file.

It may be appreciated that features, like beat shapes, may be estimated from the decompressed file. Specifically, the decompressed file contains a waveform. A section of the waveform which contains the feature may be extracted and fed into a classifier. The classifier has models for various types of standard beat shapes (some may be normal and others may be abnormal), and processes the extracted section of the waveform to determine which standard beat shape it is closest to. The classifier then proceeds to annotate the extracted section of the waveform according to at least one of the determined standard beat shapes.

In some embodiments, the processor 122 may detect a predetermined event in the decompressed data file, and insert at least one annotation associated with the detected predetermined event as the set of information.

In some embodiments, the processor 122 may detect the predetermined event based on presence or absence of at least one feature estimated from the decompressed data file. In some embodiments, the processor 122 may process the decompressed data file and classify the decompressed data file as an instance of the predetermined event. The processor 122 may insert the annotation associated with the detected predetermined event. The annotation may be located at a portion of the dataset where the predetermined event occurred. The set of information generated by the processor 122 may contain the annotation of the detected predetermined event.

In some embodiments, the processor 122 may analyse a trend in an activity of the person's body associated with the physiological parameter. The set of information generated by the processor 122 may contain the analysed trend.

In some embodiments, the processor 122 may classify the decompressed data file into at least one preliminary group of the medical condition based on the detected predetermined event and/or the analysed trend. The set of information generated by the processor 122 may contain the classification result.

The processor 122 may then compress the set of information in a compressed format and generate a compressed data file (hereinafter referred to as a “second compressed data file”). In some embodiments, the communication module 121 can send the second compressed data file to the client device 130. It may be appreciated that since the second compressed data file does not contain the dataset, the data volume of the second compressed data file may be smaller than the data volume of the first compressed data file.

The client device 130 may be a communication device used by a user, for example a doctor such as a cardiac specialist. The doctor may be employed by the system 100 or contracted with the system 100 to provide the server 120 with a verification of the the set of information received from the server 120 and generate further information associated with the dataset.

The client device 130 may receive the second compressed data file from the server 120, and decompress the second compressed data file to display the set of information on a screen, for example on a user interface. Thereafter, the client device 130 may decompress a part of the first compressed data file which is associated with a selected part of the set of information, and display at least a part of the dataset or the data corresponding to the part of the first compressed data file on the user interface.

In this manner, the client device 130 may display at least the part of the dataset and the set of information for the doctor to verify the set of information and generate further information. In this manner, the doctor may refer to the set of information in relation to the interested part of the dataset when he verifies the set of information including the classification result of the dataset which has been determined by the server 120.

More specifically, in some embodiments, the client device 130 may decompress the second compressed data file and display the set of information on the screen. The doctor may select a part of the set of information for further details. The client device 130 may determine a relevant part of the first compressed data file corresponding to the selected part of the set of information. Thereafter, the client device 130 may decompress the relevant part of the first compressed data file and display at least a part of the dataset corresponding to the relevant part of the first compressed data file which has been decompressed.

In some embodiments, the set of information can be displayed together with the part of the dataset corresponding to the part of the first compressed data file. For example, the client device 130 may overlay the annotation on the part of the dataset. The annotation may be overlaid at a portion of the dataset where the predetermined event occurred. In this manner, the client device 130 can decompress the part of the first compressed data file, on as-needed basis.

It may be appreciated that the client device 130 may process the first compressed data file and/or the second compressed data file. For example, if the doctor may wish to change the information, for example an annotation, the doctor may input the changed annotation into the client device 130. The client device 130 may receive the input to change the annotation, re-compute the corresponding information, and update the same. The client device 130 may also send the updated information to the server 120. In this regard, the reverse mapping can be performed by the client device 130 in order to improve response time.

In some embodiments, the client device 130 may generate a final report based on the set of information. In some embodiments, the client device 130 may assist in the preparation of a customized final report based on the doctor's verification of the set of the information and further information. It may be appreciated that the client device 130 may refer to a preference of another party, for example person, guardian or family doctor, who will receive the final report.

The database 123 is operable to store at least one of the following information: the dataset collected from the person, the information generated by the processor 122, verified information and generated further information, and the final report. The processor 122 may comprise non-transitory computer readable medium for executing at least one method for [x] in the form of an algorithm. In some embodiments, the at least one algorithm may be stored in an external server, so the server 120 may access the algorithm using a web service call.

Although not shown, the system 100 may further comprise at least one communication device used by another party, for example person, guardian or family doctor. The person, guardian or family doctor may receive the verification of the set of the information and the further information from the server 120. For example, the communication device may receive the final report from the server 120.

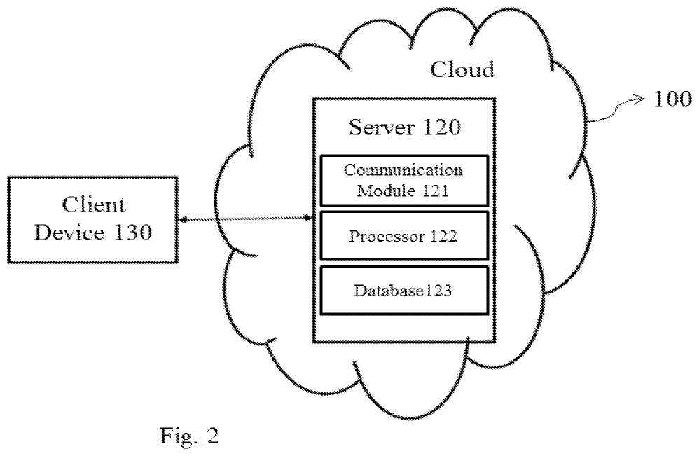

FIG. 2 shows another block diagram in accordance with some embodiments of the present invention. The system 100 may comprise a server 120 and a client device 130. In some embodiments, the client device 130 may be integrated with a device 110 described above with FIG. 1. For example, the client device 130 may include an integral data collection module (not shown). The client device 130 may include, but not be limited to, a home medical equipment used for the person at home or other facilities.

The home medical equipment as the client device 130 may collect the dataset associated with the physiological parameter of the person, compress the dataset to generate the first compressed data file, and send out the first compressed data file to the server 120. In some embodiments, the client device 130 may send the first compressed data file to the server 120 in real-time or near real-time. For example, the client device 130 may delete the dataset once the first compressed data file is sent to the server 120. In another example, the client device 130 may delete the dataset once the first compressed data file is generated.

The client device 130 may then receive the set of information in a compressed format, i.e. the second compressed data file, from the server 120. The client device 130 can process the second compressed data file to display the set of the information. More specifically, the client device 130 may decompress the second compressed data file and display the set of information on the screen. The doctor may select a part of the set of information for further details. The client device 130 may determine a relevant part of the first compressed data file corresponding to the selected part of the set of information. Thereafter, the client device 130 may decompress the relevant part of the first compressed data file and display at least a part of the dataset or data corresponding to the relevant part of the first compressed data file which has been decompressed.

As another example, the client device 130 may store the dataset even after the first compressed data file is sent to the server 120. The client device 130 may receive the set of information in the compressed format, i.e. the second compressed data file, decompress the second compressed data file, and then combine the stored dataset with the received set of information. The client device 130 may display the dataset and the set of information together.

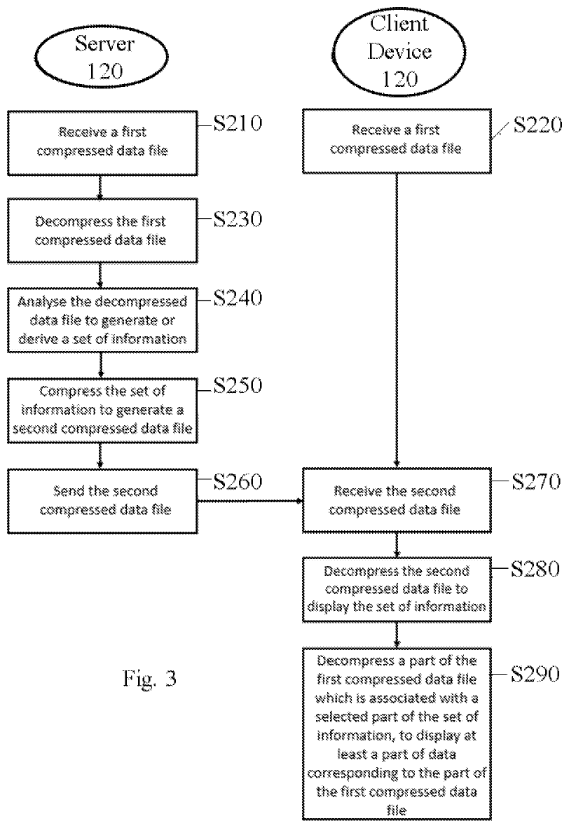

FIG. 3 shows a flow diagram in accordance with some embodiments of the present invention.

First, the server 120 may receive a first compressed data file associated with a physiological parameter of a person (S210). In some embodiments, the device 110 may detect signals from the person's body and generate the dataset associated with the physiological parameter of the person. Thereafter, the device 110 may compress the dataset to generate the first compressed data file, and send the first compressed data file to the server 120. In some other embodiments, the client device 130 may detect signals from the person's body and generate dataset associated with the physiological parameter of the person. Thereafter, the client 130 may compress the dataset to generate the first compressed data file, and send the first compressed data file to the server 120.

The client device 130 may receive the first compressed data file for the dataset associated with the physiological parameter of the person (S220). In some embodiments, the client device 130 may receive the first compressed data file from the device 110. In some other embodiments, the client device 130 may receive the first compressed data file from the server 120. In some other embodiments, the client device 130 may include an integral data collection module (not shown) as described above with FIG. 2, and receive the first compressed data file from the data collection module. Although not shown, the client device 130 may receive the dataset from the data collection module and compress the dataset to generate the first compressed data file. In an example, the client device 130 may delete the dataset once the first compressed data file is sent to the server 120. In another example, the client device 130 may delete the dataset once the first compressed data file is generated.

In some embodiments, the device 110 may continuously collect the dataset associated with the physiological parameter from the person, and generate the first compressed data file in real-time or near real-time. In some embodiments, the device 110 may generate a plurality of first compressed data files. In some other embodiments, the device 110 may send the first compressed data file to the server 120 and/or the client device 130 after a predetermined time, for example few days or few weeks. The server 120 and/or the client device 130 may then receive the first compressed data file for the dataset associated with the physiological parameter of the person.

The server 120 may decompress the first compressed data file (S230) and analyse the decompressed data file using at least one algorithm to generate or derive a set of information associated with the person (S240).

In some embodiments, the server 120 may analyse the decompressed data file, for example the ECG waveforms, using the algorithm to identify at least one of the following:
Rhythms (e.g. various types of tachycardia, sinus, atrial fibrillation, bigeminy, trigeminy, blocks, pauses, placed rhythms, ventricular fibrillation, etc.)
Sections which are unanalysed due to missing data or which are poor quality due to noise.
Key events including periods when the highest or lowest heart rate was detected or the longest or fastest runs of a particular beat shape was observed, RR-interval or NN-interval, the largest ST-segment deviations on each lead, etc.
Isolated beats, couplets and runs with a bundle branch block, premature atrial/ventricular contractions, etc.

In some embodiments, the server 120 may detect a predetermined event in the decompressed data file, and insert at least one annotation associated with the detected predetermined event as the set of information.

In some embodiments, the server 120 may detect the predetermined event based on presence or absence of at least one feature estimated from the dataset. In some embodiments, the processor 122 may process the decompressed data file and classify the decompressed data file as an instance of the predetermined event. Therefore, the set of information generated by the server 120 may contain the annotation.

In some embodiments, the server 120 may analyse a trend in an activity of the person's body associated with the physiological parameter. The set of information generated by the server 120 may contain the analysed trend.

In some embodiments, the server 120 may classify the decompressed data file into at least one preliminary group of the medical condition based on the detected predetermined event and/or the analysed trend. The set of information generated by the server 120 may contain the classification result.

The server 120 may then compress the set of information and generate a second compressed data file using the set of information (S250), and then provide the second compressed data file to the client device 130 for a display (S260). The client device 130 may receive the second compressed data file (S270) and decompress the second compressed data file to display the set of information (S280).

The amount of the dataset, for example ECG waveforms, obtained in a long term may be large (e.g. a few GB). In some embodiments, the server 120 may provide the first compressed data file for the dataset to minimize the resource requirement on the client device 130. In some other embodiments, the device 110 may provide the first compressed data file for the dataset to minimize the resource requirement on the client device 130. In this manner, the client device 130 may store the first compressed data file, not the dataset.

The client device 130 may process the first compressed data file directly, by decompressing the first compressed data file, on as-needed basis. More specifically, the doctor may select a part of the displayed set of information for further details. The client device 130 may determine a relevant part of the first compressed data file corresponding to the selected part of the set of information. The client device 130 may decompress the part of the first compressed data file which is associated with a selected part of the set of information, and display at least a part of the dataset or data corresponding to the part of the first compressed data file on the user interface (S290).

It may be appreciated that some formats, for example MIT format, may allow the doctor to access specific portions of the ECG waveform, based on start and end time and/or leads representing an interested part of the heart.

In some embodiments, the client device 130 may process the first compressed data file to render the dataset, for example the ECG waveform, and overlay the annotations on the ECG waveform.

In some embodiment, if the first compressed data file or the second compressed data file is not compatible with the client device 130, the client device 130 is operable to inform the server 120, and the server 120 is operable to convert a format of the first compressed data file or the second compressed data file into a predetermined format which is compatible with the client device 130.

In some embodiments, if the client device 130 obtains a data file which is not compatible with the client device 130, from any other device, the client device 130 is operable to upload the obtained data file to the server 120, and the server 120 is operable to convert the data file to be in a predetermined format which is compatible with the client device 130. In this regard, the server 120 may allow the client device 130 to process the data file directly.

In this manner, the system 100 can provide at least one of the following advantages:
A compressed version of the dataset, for example ECG waveform, may be communicated between the server 120 and the client device 130.
The resource requirements at the client device 130 may remain minimal as the client device 130 can process the first compressed data file with minimal decompression.
A file access may be improved, leading to overall higher rendering speed at the client device 130, without latency.

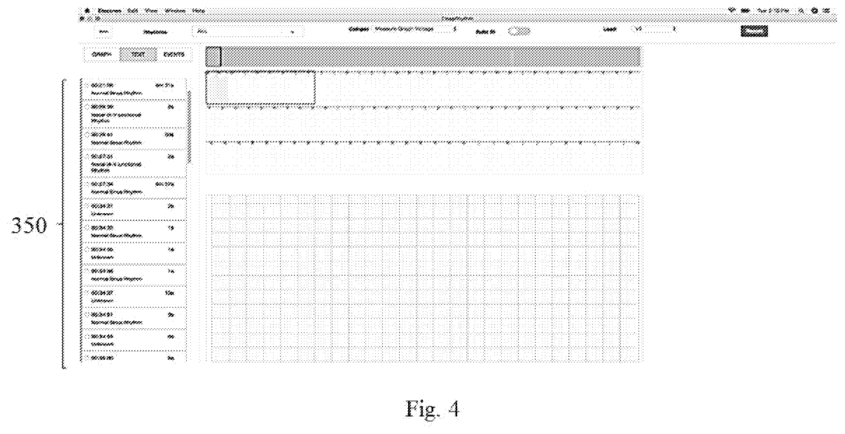

FIGS. 4 to 6 show examples of information displayed in the client device 130 in accordance with some embodiments of the present invention.

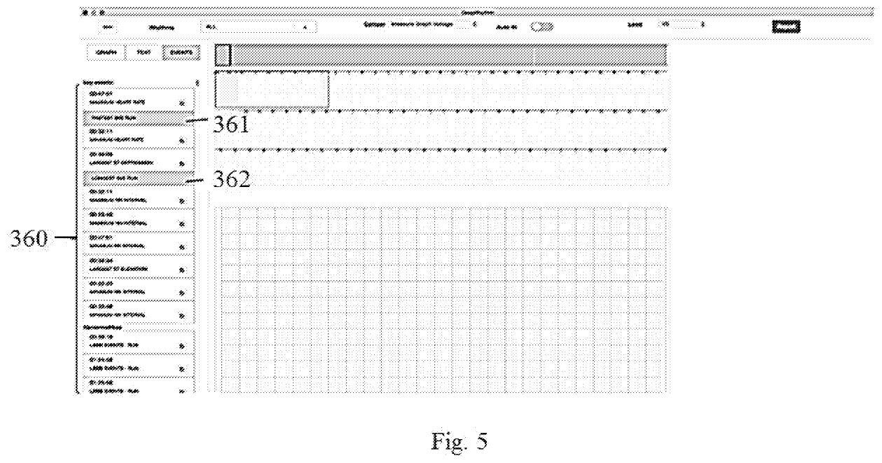

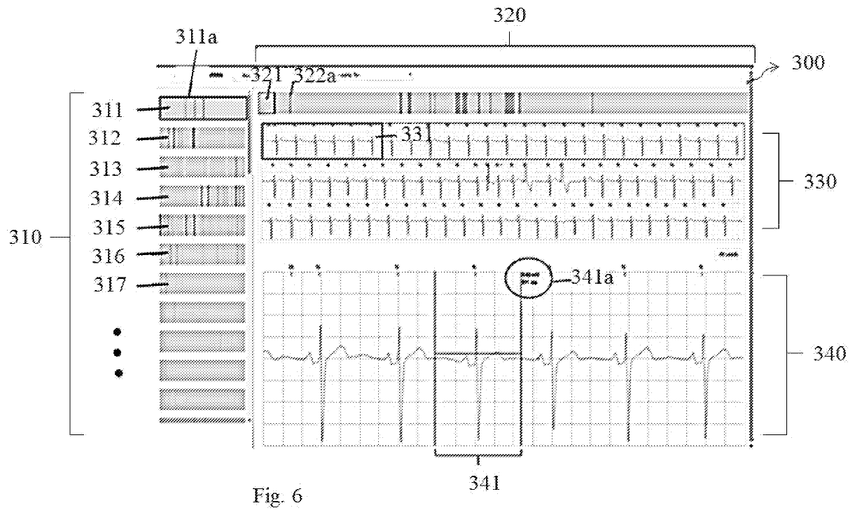

The decompressed part of the first compressed data file, for example the decompressed part of the dataset, and the information generated by the server 120 may be displayed in the client device 130. The information may assist a user, for example a doctor, in accomplishing a series of tasks to verify the set of information and generate further information associated with the dataset. Overall, there may be two view modes as follows:
List view: In this view, a list of episodes of each type of finding (e.g. arrhythmia, erroneous sections, key events, couplets and/or runs) may be displayed along with time when they occurred. The list view may include a text view and an event view. In the text view, the list of episodes 350 may be displayed along with time when they occurred, as shown in FIG. 4. In the event view, as shown in FIG. 5, the list of episodes 360 are classified based on the type of event, for example fastest SVE (Supraventricular Ectopic Beat) run 361 and longest SVE run 362. The user can select any of these episodes by click or touch input to jump to an appropriate portion of the ECG waveforms in the graph view.
Graph view: In this view, the ECG waveforms' tracing may be displayed in different levels of zoom. This view may allow the user to view the ECG waveforms along with the algorithmic findings (e.g. annotations) to make his final diagnosis, as shown in FIG. 6.

More specifically, as shown in FIG. 6, the client device 130 may split the first compressed data file into a plurality of groups based on a predetermined time interval. The client device 130 may display the plurality of groups of the dataset on the screen as a plurality of decompressed data strips 310. It may be appreciated that a predetermined event which occurred may be indicated as a colour code 311a, 322a. For example, the colour code may vary depending on the seriousness of the predetermined event.

If a decompressed data strip 311 among the plurality of decompressed data strips 310 is selected, the client device 130 may display an extended data strip 320 corresponding to the selected decompressed data strip 311 on the screen. If a region 321 in the extended data strip 320 is selected, the client device 130 may decompress a part of the first compressed data file corresponding to the selected region 321 of the extended data strip 320 and display decompressed waveforms 330 corresponding to the selected region 321 on the screen. If a region 331 in the decompressed waveforms 330 is selected, the client device may display extended waveforms 340 corresponding to the selected region 331 of the decompressed waveforms 330 on the screen.

The annotation may be displayed on the decompressed waveforms 330 and/or extended waveforms 340 to indicate locations and type of beats. For example, an abbreviation or initial with respect to the annotation may be shown on top of each beat. As shown in FIG. 6, a portion 341 of the extended waveforms 340 which may be of interest, for example due do the occurrence of the event, may be measured, for example in terms of time or voltage between edges of the box, using the virtual calipers.

In some embodiments, the user can directly navigate to a region of interest (e.g. rhythms, erroneous section, key events, couplets and/or runs) from the screen of the client device 130 using selection tools provided in a portion the screen, for example at the top of the graph view. The user may also navigate to these sections from the text view shown in FIG. 4 or the event view shown in FIG. 5.

In some embodiments, the navigation mechanism may allow the user to first study the atrial fibrillation episodes, for example with all other rhythm annotations removed, go from an episode to an episode of this rhythm and if necessary correct any specific episode or all episodes, before moving on to the episodes of ventricular fibrillation rhythm in the second pass. In any of these passes, the user can use different views to see morphology of beats in the surrounding regions or use the inbuilt calipers in another view to make detailed measurements. During this review if the user changes a rhythm, the underlying beat annotations and the dependent summary calculations are updated automatically by at least one of the server 120 or the client device 130. For example, the best annotations may be updated by the client device 130 and the summary calculations may be updated by the server 120.

In some embodiments, as shown in FIG. 6, the graph view mode may include the following sub-view modes:
First view (Bird's eye view): It may be appreciated that the decompressed data strips 310 and the extended data strip 320 are part of the first view, as in the first view the actual waveforms may not be shown. This view may show the occurrence of events and/or rhythms with respect to time. For example, this view may provide a colour-coded representation of the entire ECG waveforms. This view may not plot the actual ECG waveforms. Instead, this view may simply provide colour-codes sections based on the occurrence of the event, for example arrhythmia, analysed by the server 120. The ECG waveforms may not need to be read in this view, since this view can be generated from the set of information, for example annotations. This view may provide the user with a quick temporal view of the rhythms seen in the person's recording. This view may provide the user with an idea of the temporal distribution of the rhythms and their frequency of occurrence. If other sensor data such as activity or SpO2 are available, this view may show an overview of each parameter. For example the sensor readings can be separated into meaningful non-overlapping categories, and the temporal distribution of the categories can be shown in this view. This may allow the user to see how the various sensor readings are correlated with the ECG waveforms. The user can click on any section to see an expanded view (with ECG waveforms) in the second and third views. The actual ECG waveforms may be shown in the second and third views.
Second view: This view may show the ECG waveforms and be used by the user to verify the rhythm of the ECG waveforms. The annotations may be marked on this view.
Third view: This view may show an expanded view of the ECG waveforms and be used to make measurements, using the calipers functionality provided. There are two modes in which the calipers can be used. The first mode may be used to find the distance between two points, while the second mode may be used to first pre-set the width between the two points, and then move it across the beats to check if the distance between two points (e.g. distance between P and QRS waves) within a beat or the distance between adjacent beats (e.g. R waves between two adjacent beats) is the same from beat to beat.

The user can choose the lead to be shown in the second and third views.

An additional sub-view mode which shows a subsampled version of the rhythm lead may be provided to allow the user to scan through the entire ECG waveforms (without delving into details) for sanity check. By subsampling, certain ECG features, for example the high frequency components, may be lost, but the ECG rendering may be sped up, lowering the latency. This view may be useful in terms of lowering the latency, since the user may use this view for sanity check.

Another sub-view mode available to the user may be the all-lead view in which all the available lead dataset is displayed simultaneously. The user may choose to use this view to verify findings from the rhythm lead, by reviewing the dataset from other leads.

Conventional software has not allowed the user to quickly locate objects of interest in the dataset (e.g. the systems have not been designed to quickly locate three frames with dogs in a 24-hour video stream dataset). The task oriented viewer design coupled with the algorithms may achieve this objective. Some embodiments may apply to other industries which use a hybrid (human-computer) model to locate infrequently occurring patterns in large volumes of dataset.

It may be appreciated by the person skilled in the art that variations and combinations of features described above, not being alternatives or substitutes, may be combined to form yet further embodiments falling within the intended scope of the invention.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A system for displaying physiological information comprising:
a server arranged to receive a first compressed data file associated with a physiological parameter of a person, decompress the first compressed data file, and analyze the decompressed data file to generate or derive a set of information associated with the person; and
a client device configured to receive the first compressed data file, and communicate with the server to receive the set of information for display on a user interface;
wherein the server is configured to compress the set of information to generate a second compressed data file, and send the second compressed data file to the client device; and
the client device configured to receive the second compressed data file from the server, decompress the second compressed data file to display the set of information on the user interface, and decompress a part of the first compressed data file which is associated with a selected part of the set of information, to display at least a part of data corresponding to the part of the first compressed data file on the user interface.

2. The system according to claim 1, wherein the analysis of the decompressed data file comprises a comparison between at least one feature extracted from the decompressed data file and a predetermined physiological parameter.

3. The system according to claim 2, wherein the analysis of the decompressed data file comprises a detection of a predetermined event in the decompressed data file, and the set of information contains at least one annotation associated with the detected predetermined event.

4. The system according to claim 3, wherein the detection of the predetermined event is performed based on presence or absence of at least one feature estimated from the decompressed data file.

5. The system according to claim 3, wherein the detection of the predetermined event comprises processing of the decompressed data file and a classification of the decompressed data file as an instance of the predetermined event.

6. The system according to claim 2, wherein the analysis of the decompressed data file comprises an analysis of trend in an activity of the person's body associated with the physiological parameter, and the set of information contains the analyzed trend.

7. The system according to claim 6, wherein the analysis of the decompressed data file comprises a classification of the decompressed data file into at least one preliminary group of medical condition based on the detected predetermined event and/or the analyzed trend.

8. The system according to claim 1, wherein the client device is operable to split the first compressed data file into a plurality of groups based on a predetermined time interval.

9. The system according to claim 8, wherein the client device is operable to display a plurality of decompressed data strips on the user interface.

10. The system according to claim 9, wherein if a data strip among the plurality of decompressed data strips is selected, the client device is operable to display an extended data strip corresponding to the selected decompressed data strip on the user interface.

11. The system according to claim 10, wherein if a region in the extended data strip is selected, the client device is operable to decompress a part of the first compressed data file corresponding to the selected region of the extended data strip and display at least a part of decompressed waveforms corresponding to the selected region on the user interface.

12. The system according to claim 11, wherein if a region in the decompressed waveforms is selected, the client device is operable to display extended waveforms corresponding the selected region of the decompressed waveforms on the user interface.

13. The system according to claim 12, wherein the annotation is displayed on the decompressed waveforms and/or extended waveforms.

14. The system according to claim 1, wherein if at least one of the first or second compressed data file is not compatible with the client device, the client device is operable to inform the server, and the server is operable to convert a format of the at least one of the first or second compressed data file to be compatible with the client device.

15. The system according to claim 1, wherein if the client device obtains another data file which is not compatible with the client device, the client device is operable to send the another data file to the server, and the server is operable to convert the another data file to be compatible with the client device.

16. The system according to claim 1, wherein if the client device an input to change the information, the client device updates the information based on the input and sends the updated information to the server.

17. A method for displaying physiological information comprising:
receiving, at a server and a client device, a first compressed data file associated with a physiological parameter of a person;
decompressing, at the server, the first compressed data file;
analysing, at the server, the decompressed data file to generate or derive a set of information associated with the person;
compressing, at the server, the set of information to generate a second compressed data file;
sending, at the server, the second compressed data file to the client device;
receiving, at the client device, the second compressed data file from the server;
decompressing, at the client device, the second compressed data file to display the set of information on the user interface; and
decompressing, at the client device, a part of the first compressed data file which is associated with a selected part of the set of information, to display at least a part of data corresponding to the part of the first compressed data file on the user interface.
